(19) United States
(12) Patent Application Publication
(10) Pub. No.: US 2005/0267030 A1
(43) Pub. Date: Dec. 1, 2005

(54) Use of deltaPKC peptides for modulation of reactive oxygen species

(76) Inventors: Philip S. Tsao, Los Altos, CA (US); Daria Mochly-Rosen, Menlo Park, CA (US)

Correspondence Address: PERKINS COIE LLP, P.O. BOX 2168, MENLO PARK, CA, 94026, US

(21) Appl. No.: 11/119,098

(22) Filed: Apr. 29, 2005

Related U.S. Application Data
(60) Provisional application No. 60/567,315, filed on Apr. 30, 2004.

(51) Int. Cl. A61K 38/18; A61K 38/48

(52) U.S. Cl. 514/12; 424/94.5

(57) ABSTRACT

Methods for modulating the level of reactive oxygen species (ROS) in a cell or tissue by administering compounds that modulate the activity of δPKC are described. In one embodiment, the level of ROS is decreased by administering a peptide effective to inhibit the activity of δPKC. In another embodiment, the level of ROS is increased by administering a peptide having activity to agonize the activity of δPKC. Also described are methods to treat or inhibit development of conditions preceded by or exacerbated by an increased level of ROS, such as arteriosclerosis and inflammatory diseases.

This application claims the benefit of U.S. provisional application No. 60/567,315, filed Apr. 30, 2004, which is incorporated herein by reference.

### FIELD OF THE INVENTION

The present invention relates to methods of treating disorders characterized by, preceded by, or exacerbated, at least in part, by reactive oxygen species. The method includes administering a compound, such as a delta protein kinase C (δPKC) peptide, in vivo in an amount sufficient to modulate, and preferably, inhibit production of reactive oxygen species. The method finds particular use in inhibiting disorders resulting from oxidative stress, such as arteriosclerosis, reperfusion injury, arthritis, and tissue damage in diabetic subjects.

### BACKGROUND OF THE INVENTION

Species capable of independent existence that contain one or more unpaired electrons are commonly referred to as free radicals. There are many types of free radicals, differing in their reactivity, origin, place of formation, degree of lipophilicity, and potential biological target. In recent years, the term “reactive oxygen species” (ROS) has been adopted to include molecules such as hypochlorous acid (HOCI), singlet oxygen (¹O₂), superoxide anion (.O₂⁻), hydroxyl radical (. OH), and hydrogen peroxide (H₂O₂), which are not radicals in nature but are capable of radical formation in the extra- and intracellular environments (Halliwell, B. and Gutteridge, J. M. C (Eds), FREE RADICAL IN BIOLOGY AND MEDICINE, 2nd Ed. Clarendon Press, Oxford, 1989).

ROS are involved in many biological processes, including regulating biochemical processes, assisting in the action of specific enzymes, and removing and destroying bacteria and damaged cells (Halliwell, B. and Gutteridge, J. M. C (Eds), FREE RADICAL IN BIOLOGY AND MEDICINE, 2nd Ed. Clarendon Press, Oxford, 1989). Free radicals are essential for the body and under normal circumstances there is a balance between oxidative and reductive compounds (redox state) inside the cell. If the balance is impaired in favor of oxidative compounds, oxidative stress is said to occur (Parke et al., Int. J. Occup. Med. Environ. Health 9:331-340 (1996); Knight, Ann. Clin. Lab. Sci. 27:11-25 (1997); Stohs, J. Basic. Clin. Physiol. Pharmacol. 6: 205-228 (1995)). Oxidative stress may occur as a result of oxidative insults such as air pollution or the “oxidative burst” characteristic of activated neutrophils mediated by the immune response. A constant source of oxidative stress results from formation of superoxide anion via “electron leakage” in the mitochondria during production of adenosine triphosphate (ATP). Although superoxide anion is not exceedingly reactive in and of itself, it can initiate a chain of events that eventually results in the formation of the highly reactive free radicals and other oxidants. If these reactive oxygen species are not controlled by enzymatic and/or non-enzymatic antioxidant systems, in vivo oxidation of critical cellular components such as membranes, DNA, and proteins will result, eventually leading to tissue damage and dysfunction.

Reactive oxygen species (ROS) have been implicated in the development of many disorders. ROS are involved in artherosclerotic lesions, in the evolution of various neurodegenerative diseases, and are also produced in association to epileptic episodes, in inflammation, in the mechanisms of action of various neurotoxicants, or as side effects of drugs. ROS also appear to play a role in reperfusion injury.

It is clear that the balance between oxidative and reductive compounds in biological systems is important. To preserve this balance the body has a number of protective antioxidant mechanisms that remove or prevent formation of ROS. There are also mechanisms that repair damage caused by ROS in vivo. Defense systems include enzymatic as well as non-enzymatic antioxidant components. However, the development of methods and compounds to combat oxidative stress or toxicity associated with oxygen-related species has enjoyed limited success. Thus, there remains a need in the art for therapies that provide a defense against damage due to reactive oxygen species.

Protein kinase C (PKC) is a key enzyme in signal transduction involved in a variety of cellular functions, including cell growth, regulation of gene expression and ion channel activity. The PKC family of isozymes includes at least eleven different protein kinases which can be divided into at least three subfamilies based on their homology and sensitivity to activators. Members of the classical or cPKC subfamily, α, β_{I}, β_{II} and γPKC, contain four homologous domains (C1, C2, C3 and C4) inter-spaced with isozyme-unique (variable or V) regions, and require calcium, phosphatidylserine (PS), and diacylglycerol (DG) or phorbol esters for activation. Members of the novel or nPKC subfamily, δ, ε, η, and θPKC, lack the C2 homologous domain and do not require calcium for activation. Finally, members of the atypical or αPKC subfamily, ζ and NiPKC, lack both the C2 and one half of the C1 homologous domains and are insensitive to DG, phorbol esters and calcium.

Studies on the subcellular distribution of PKC isozymes demonstrate that activation of PKC results in its redistribution in the cells (also termed translocation), such that activated PKC isozymes associate with the plasma membrane, cytoskeletal elements, nuclei, and other subcellular compartments (Saito, N. et al., Proc. Natl. Acad. Sci. USA 86:3409-3413 (1989); Papadopoulos, V. and Hall, P. F. J. Cell Biol. 108:553-567 (1989); Mochly-Rosen, D., et al., Molec. Biol. Cell (formerly Cell Reg.) 1:693-706 (1990)).

The localization of different PKC isozymes to different areas of the cell in turn appears due to binding of the activated isozymes to specific anchoring molecules termed Receptors for Activated C-Kinase (RACKs). RACKs are thought to function by selectively anchoring activated PKC isozymes to their respective subcellular sites. RACKs bind only fully activated PKC and are not necessarily substrates of the enzyme. Translocation of PKC reflects binding of the activated enzyme to RACKs anchored to the cell particulate fraction and the binding to RACKs is required for PKC to produce its cellular responses (Mochly-Rosen, D., et al., Science 268:247-251 (1995)).

### SUMMARY OF THE INVENTION

Accordingly, the invention to provides a method of modulating the level of reactive oxygen species (ROS) in a cell or tissue.

The invention also provides a method of decreasing the level of ROS in a cell or tissue.

The invention also provides a method of increasing the level of ROS in a cell or tissue.

The invention also provides a method of reducing or protecting cells and tissue from damage due to ROS.

The invention also provides a method of increasing or decreasing the activity of δPKC to modulate the level of ROS in a cell or tissue.

The invention also provides a method of treating a disorder associated with oxidative stress, i.e., and increased level of ROS in a particular tissue.

In one aspect, the invention includes a method of modulating production of reactive oxygen species in a cell or tissue by administering a compound effective to modulate the activity of δPKC. The compound in one embodiment has activity to inhibit δPKC activity to reduce production of reactive oxygen species in the cell or tissue. In another embodiment, the compound has activity to activate δPKC activity to stimulate production of reactive oxygen species in the cell or tissue.

Compounds having inhibitory activity are, in one embodiment, isolated peptides having at least 50% sequence identity to a peptide selected from the group consisting of δV1-1 (SEQ ID NO:3), δV1-2 (SEQ ID NO:4), and δV1-5 (SEQ ID NO:6). The peptide can also be selected from the group consisting of SEQ ID NO:4 ( δV1-1) and the peptides identified as SEQ ID NOS:33-47. The peptide can also be selected from the group consisting of δV1-2 (SEQ ID NO:4) and the peptides identified as SEQ ID NOS:64-70.

Compounds having activity to activate PKC are in one embodiment a peptide having at least 50% sequence identity to ψδRACK (SEQ ID NO:5). Such peptides include peptides selected from the group consisting of ψδRACK (SEQ ID NO:5) and the peptides identified as SEQ ID NOS:10-18, 21-32.

The peptides can be conjugated with a moiety to facilitate transfer across a cell membrane. The peptides can be administered by any suitable route including but not limited to intravenous, parenteral, subcutaneous, inhalation, intranasal, sublingual, mucosal, and transdermal route. In one embodiment, the peptide is administered during a period of reperfusion; that is, after a period of initial perfusion.

In another aspect, the invention includes a method for inhibiting development of, slowing the progression of, or treating arteriosclerosis by administering a compound effective to inhibit the activity of δPKC. The compound is preferably a peptide having at least 50% sequence identity to a peptide selected from the group consisting of δV1-1 (SEQ ID NO:3), δV1-2 (SEQ ID NO:4), and δV1-5 (SEQ ID NO:6). The peptide can be conjugated to a moiety to assist transport across a cell membrane.

In another aspect, the invention includes method for treating a disorder characterized by or preceded by an increased production of reactive oxygen species in a cell or tissue by administering a compound effective to inhibit the activity of δPKC. In one embodiment, the compound is a peptide having at least 50% sequence identity to a peptide selected from the group consisting of δV1-1 (SEQ ID NO:3), δV1-2 (SEQ ID NO:4), and δV1-5 (SEQ ID NO:6). The peptide can be conjugated to a cell membrane transport moiety, such as Tat. The method finds use, for example, in inflammatory disorders, such as arthritis, and vascular disorders.

In another aspect, the invention includes a method for stimulating production of reactive oxygen species in a cell or tissue, by administering a compound effective to agonize the activity of δPKC. In one embodiment, the compound is a peptide having at least 50% sequence identity to ψδRACK (SEQ ID NO:5). In another embodiment, the peptide is administered to a tumor cell. The agonist peptide can be linked to a moiety effective to facilitate transport across a cell membrane.

These and other objects and features of the invention will be more fully appreciated when the following detailed description of the invention is read in conjunction with the accompanying drawings.

### BRIEF DESCRIPTION OF THE DRAWINGS

FIG. 1 shows the alignment of the primary sequence of rat δPKC and mouse ΘPKC V1 domains. The bracketed areas designated as δV1-1, δV1-2, and ψδR indicate regions of difference between the two isozymes.

FIG. 2 shows the production of reactive oxygen species (ROS), in fmols/mg protein, in rat vascular smooth muscle cells in vitro, after incubation in the presence of tempoamine alone (control) or with ψδRACK, phorbol 12-myristate 13-acetate (PMA), and δV1-1 plus PMA;

FIG. 3A shows the production of reactive oxygen species (ROS), in fmols/mg protein, in Zucker Lean rats (Fa/fa), Zucker obese rats (fa/fa), and Zucker obese rats treated with insulin-sensitizing thiazolidinedione;

FIGS. 3B-3C is a graph showing the stress, in kPa, as a function of strain, in m/m, of aortas excised from Zucker lean rats (lower curve) and Zucker obese (upper curve) rats in the circumferential (FIG. 3B) and longitudinal (FIG. 3C) directions;

FIG. 4A shows a Western blot autoradiogram of the cytosol and membrane cell fractions of rat vascular smooth muscle cells incubated with bovine insulin and probed with anti-δPKC;

FIG. 4B is a bar graph showing the relative δPKC activity, based on the Western blot of FIG. 4A, in cells incubated with bovine insulin, the δPKC activity normalized against the δPKC activity in the control cells;

FIG. 5A is a bar graph showing the normalized tumor growth factor-beta (TGF-β) expression in vitro in rat vascular smooth muscle cells left untreated (control) and incubated with insulin

FIG. 5B is a bar graph showing the normalized TGF-β expression in vitro in rat vascular smooth muscle cells left untreated (control) and incubated with Tat-conjugated δV1-1 peptide;

FIG. 5C is a bar graph showing the normalized Cbfa-1 gene expression in vitro in rat vascular smooth muscle cells left untreated (control) and incubated with Tat-conjugated δV1-1 peptide; and

FIG. 6 is a bar graph showing the increase in osteopontin expression (expressed as percent of control) in vivo in Zucker obese rats relative to Zucker lean rats.

### BRIEF DESCRIPTION OF THE SEQUENCES

SEQ ID NO:1 corresponds to amino acids 1-141 from the V1 domain of rat δPKC (accession no. KIRTCD).

SEQ ID NO:2 corresponds to amino acids 1-124 of V1 domain of mouse theta-PKC (accession no. Q02111).

SEQ ID NO:3 is an amino acid sequence from the first variable region of δPKC (amino acids 8-17), δV1-1.

SEQ ID NO:4 is an amino acid sequence from the first variable region of δPKC (amino acids 35-44), δV1-2.

SEQ ID NO:5 is an amino acid sequence from δPKC (amino acids 74-81), and is referred to herein as “pseudo-delta” RACK, or ψδRACK.

SEQ ID NO:6 is an amino acid sequence from a region of δPKC (amino acids 619-676), referred to herein as δV1-5.

SEQ ID NO:7 is the Drosophila Antennapedia homeodomain-derived carrier peptide.

SEQ ID NO:8 is a Tat-derived carrier peptide (Tat 47-57).

SEQ ID NO:9 is a βPKC-selective activator peptide.

SEQ ID NO:10 is a modification of SEQ ID NO:5 (ψδRACK).

SEQ ID NO:11 is a modification of SEQ ID NO:5 (ψδRACK).

SEQ ID NO:12 is a modification of SEQ ID NO:5 (ψδRACK).

SEQ ID NO:13 is a modification of SEQ ID NO:5 (ψδRACK).

SEQ ID NO:14 is a modification of SEQ ID NO:5 (ψδRACK).

SEQ ID NO:15 is a modification of SEQ ID NO:5 (ψδRACK).

SEQ ID NO:16 is a modification of SEQ ID NO:5 (ψδRACK).

SEQ ID NO:17 is a modification of SEQ ID NO:5 (ψδRACK).

SEQ ID NO:18 is a modification of SEQ ID NO:5 (ψδRACK).

SEQ ID NO:19 is a fragment of SEQ ID NO:5 (ψδRACK).

SEQ ID NO:20 is a fragment of SEQ ID NO:5 (ψδRACK).

SEQ ID NO:21 is a modification of SEQ ID NO:5 (ψδRACK).

SEQ ID NO:22 is a modification of SEQ ID NO:5 (ψδRACK).

SEQ ID NO:23 is a modification of SEQ ID NO:5 (ψδRACK).

SEQ ID NO:24 is a modification of SEQ ID NO:5 (ψδRACK).

SEQ ID NO:25 is a modification of SEQ ID NO:5 (ψδRACK).

SEQ ID NO:26 is a modification of SEQ ID NO:5 (ψδRACK).

SEQ ID NO:27 is a modification of SEQ ID NO:5 (ψδRACK).

SEQ ID NO:28 is a modification of SEQ ID NO:5 (ψδRACK).

SEQ ID NO:29 is a modification of SEQ ID NO:5 (ψδRACK).

SEQ ID NO:30 is a modification of SEQ ID NO:5 (ψδRACK).

SEQ ID NO:31 is a modification of SEQ ID NO:5 (ψδRACK).

SEQ ID NO:32 is a modification of SEQ ID NO:5 (ψδRACK).

SEQ ID NO:33 is a modification of SEQ ID NO:3 (δV1-1).

SEQ ID NO:34 is a modification of SEQ ID NO:3 (δV1-1).

SEQ ID NO:35 is a modification of SEQ ID NO:3 (δV1-1).

SEQ ID NO:36 is a modification of SEQ ID NO:3 (δV1-1).

SEQ ID NO:37 is a modification of SEQ ID NO:3 (δV1-1).

SEQ ID NO:38 is a modification of SEQ ID NO:3 (δV1-1).

SEQ ID NO:39 is a modification of SEQ ID NO:3 (δV1-1).

SEQ ID NO:40 is a modification of SEQ ID NO:3 (δV1-1).

SEQ ID NO:41 is a modification of SEQ ID NO:3 (δV1-1).

SEQ ID NO:42 is a modification of SEQ ID NO:3 (δV1-1).

SEQ ID NO:43 is a modification of SEQ ID NO:3 (δV1-1).

SEQ ID NO:44 is a modification of SEQ ID NO:3 (δV1-1).

SEQ ID NO:45 is a modification of SEQ ID NO:3 (δV1-1).

SEQ ID NO:46 is a modification of SEQ ID NO:3 (δV1-1).

SEQ ID NO:47 is a modification of SEQ ID NO:3 (δV1-1).

SEQ ID NO:48 is a fragment of SEQ ID NO:3(δV1-1).

SEQ ID NO:49 is a modified fragment of SEQ ID NO:3 (δV1-1).

SEQ ID NO:50 is a modified fragment of SEQ ID NO:3 (δV1-1).

SEQ ID NO:51 is a modified fragment of SEQ ID NO:3 (δV1-1).

SEQ ID NO:52 is a modified fragment of SEQ ID NO:3 (δV1-1).

SEQ ID NO:53 is a modified fragment of SEQ ID NO:3 (δV1-1).

SEQ ID NO:54 is a modified fragment of SEQ ID NO:3 (δV1-1).

SEQ ID NO:55 is a modified fragment of SEQ ID NO:3 (δV1-1).

SEQ ID NO:56 is a modified fragment of SEQ ID NO:3 (δV1-1).

SEQ ID NO:57 is a fragment of SEQ ID NO:3 δV1-1.

SEQ ID NO:58 is a fragment of SEQ ID NO:3 δV1-1.

SEQ ID NO:59 is a fragment of SEQ ID NO:3 δV1-1.

SEQ ID NO:60 is a fragment of SEQ ID NO:3 δV1-1.

SEQ ID NO:61 is a fragment of SEQ ID NO:3 δV1-1.

SEQ ID NO:62 is a fragment of SEQ ID NO:3 δV1-1.

SEQ ID NO:63 is a fragment of SEQ ID NO:3 δV1-1.

SEQ ID NO:64 is a modification of SEQ ID NO:4 (δV1-2).

SEQ ID NO:65 is a modification of SEQ ID NO:4 (δV1-2).

SEQ ID NO:66 is a modification of SEQ ID NO:4 (δV1-2).

SEQ ID NO:67 is a modification of SEQ ID NO:4 (δV1-2).

SEQ ID NO:68 is a modification of SEQ ID NO:4 (δV1-2).

SEQ ID NO:69 is a modification of SEQ ID NO:4 (δV1-2).

SEQ ID NO:70 is a modification of SEQ ID NO:4 (δV1-2).

SEQ ID NO:71 is the sequence of Annexin V.

### DETAILED DESCRIPTION OF THE INVENTION

### I. DEFINITIONS

Unless otherwise indicated, all terms herein have the same meaning as they would to one skilled in the art of the present invention. Practitioners are particularly directed to Current Protocols in Molecular Biology (Ausubel, F. M. et al., John Wiley and Sons, Inc., Media Pa.) for definitions and terms of the art.

Abbreviations for amino acid residues are the standard 3-letter and/or 1-letter codes used in the art to refer to one of the 20 common L-amino acids.

A “conserved set” of amino acids refers to a contiguous sequence of amino acids that is conserved between members of a group of proteins. A conserved set may be anywhere from two to over 50 amino acid residues in length. Typically, a conserved set is between two and ten contiguous residues in length. For example, for the two peptides MKAAEDPM (SEQ ID NO:10) and MRAPEDPM (SEQ ID NO:13), there are 4 identical positions (EDPM; SEQ ID NO:19) which form the conserved set of amino acids for these two sequences.

“Conservative amino acid substitutions” are substitutions which do not result in a significant change in the activity (e.g., δV1-1 PKC activity) or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art.

“Peptide” and “polypeptide” are used interchangeably herein and refer to a compound made up of a chain of amino acid residues linked by peptide bonds. Unless otherwise indicated, the sequence for peptides is given in the order from the amino terminus to the carboxyl terminus.

Sequence “identity” is determined by comparing the amino acid sequences of polypeptides when aligned so as to maximize overlap and identity while minimizing sequence gaps. The percent identity of two amino acid or two nucleic acid sequences can be determined by visual inspection and/or mathematical calculation, commonly done for longer sequences by comparing sequence information using a computer program. An exemplary, preferred computer program is the Genetics Computer Group (GCG; Madison, Wis.) Wisconsin package version 10.0 program, ‘GAP’ (Devereux et al., Nucl. Acids Res., 12: 387 (1984)). Other programs used by those skilled in the art of sequence comparison can also be used, such as, for example, the BLAST (BLASTP) and BLASTN programs, available for use via the National Library of Medicine website http://www.ncbi.nlm.nih.gov/BLAST. In preferred embodiments, sequences are considered homologous or identical to one another if their amino acid sequences are at least about 50% identical, more preferably if the sequences are 70% or 75% identical, still more preferably if the sequences are 80% or 85% identical, still more preferably if the sequences are 90% or 95% identical, when determined from a visual inspection or from one of the aforementioned computer programs.

A peptide or peptide fragment is “derived from” a parent peptide or polypeptide if it has an amino acid sequence that is identical or homologous to the amino acid sequence of the parent peptide or polypeptide.

“Reperfusion” refers to return of fluid flow into a tissue after a period of no-flow or reduced flow. For example, in reperfusion of the heart, fluid or blood returns to the heart through the coronary arteries after occlusion of these arteries has been removed.

The term “PKC” refers to protein kinase C, or C-kinase.

The term “RACK” refers to receptor for activated C-kinase.

An “agonist” refers to endogenous substance or a drug that can interact with a receptor and initiate a physiological or a pharmacological response characteristic of that receptor (contraction, relaxation, secretion, enzyme activation, etc.)

An “antagonist” refers to molecule that opposes the physiological effects of another. At the receptor level, it is a molecule that opposes the receptor-associated responses normally induced by another bioactive agent.

### II. δPKC PEPTIDES MODULATE REACTIVE OXYGEN SPECIES

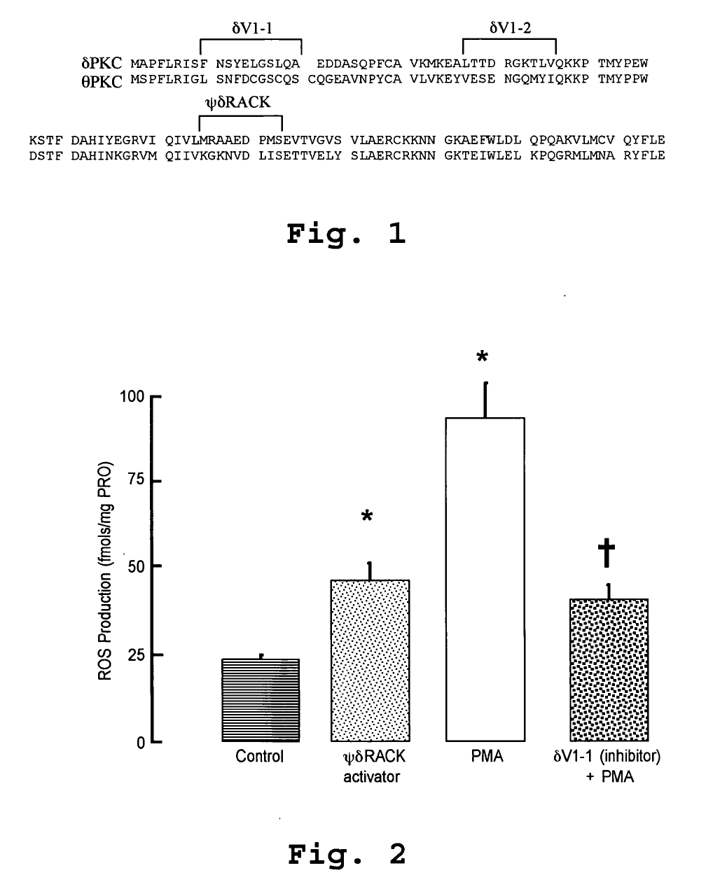

In studies described herein, peptides effective to activate (agonize) delta protein kinase C (δPKC) and peptides effective to inhibit (antagonize) δPKC were administered to cells. FIG. 1 shows the sequences of the V1 domain of rat δPKC (SEQ ID NO:1; accession no. KIRTCD) and mouse theta-PKC (Θ-PKC) V1 domain (SEQ ID NO:2, accession no. Q02111). Three regions in the V1 domain of δPKC were identified with only ˜10% identity to theta-PKC. These regions are indicated in FIG. 1 by the bars above the sequence of δPKC and are referred to herein as δV1-1 having a sequence identified herein as SEQ ID NO:3 (SFNSYELGSL), δV1-2 having a sequence identified herein as SEQ ID NO:4 (ALTTDRGKLV), and ψδRACK having a sequence identified herein as SEQ ID NO:5 (MRAAEDPM). Not shown in FIG. 1 is another sequence from the δPKC sequence identified as SEQ ID NO:6 and referred to herein as δV1-5. δV1-1, δV1-2 and δV1-5 are δPKC antagonists and ψδRACK is a δPKC agonist.

In the studies described below, the δPKC peptides were modified with a carrier peptide by cross-linking via an N-terminal Cys-Cys bond to Tat (SEQ ID NO:8). It will be appreciated that the δPKC peptides can be conjugated to other moieties to facilitate transport of the peptide across a cell membrane, for example Drosophila Antennapedia homeodomain (SEQ ID NO:7; Théodore, L. et al. J. Neurosci., 15:7158 (1995); Johnson, J. A. et al., Circ. Res. 79:1086 (1996)) or polyarginine (Mitchell et al., J. Peptide Res., 56:318-325 (2000); Rothbard et al., Nature Med., 6:1253-1257 (2000)). It will also be appreciated that the transport peptide can be conjugated to a C-terminal amino acid residue.

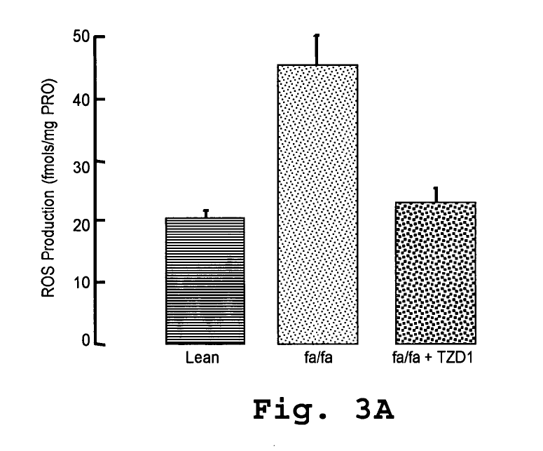

Example 1 details a study where inhibitors and activators of δPKC were administered to rat vascular smooth muscle cells in vitro. Cells were incubated in presence of (1) the agonist Tat-conjugated peptide ψδRACK (SEQ ID NO:5); (2) phorbol 12-myristate 13-acetate (PMA); or (3) the antagonist Tat-conjugated peptide δV1-1 (SEQ ID NO:3) plus PMA. A control cell sample was left untreated. Tempoamine ((2,2,6,6-tetramethyl-1-piperidi-nyloxy-4-amine)) was added to each culture and the loss of tempoamine signal was measured by electron spin resonance to measure superoxide production. The results are shown in FIG. 2.

FIG. 2 shows the production of reactive oxygen species (ROS), in fmols/mg protein, in rat vascular smooth muscle cells in vitro, after incubation. The presence of the δPKC activator, ψδRACK, resulted in an increased production of ROS. PMA, a non-isozyme specific activator of PKC, stimulated a 3-4 fold increase in ROS in the cells. Addition of the delta isozyme specific inhibitor, δV1-1 to cells co-incubated with PMA was effective to reverse or inhibit a significant part of the ROS production stimulated by PMA. These results show that that selective activation of δPKC caused a significant increase in production of ROS. Inhibition of this PKC isoform caused a decrease in PMA-stimulated ROS production.

Accordingly, a method for modulating (increasing or decreasing) production of ROS in a cell or tissue is provided, by delivering a compound effective to activate δPKC. More particularly, a compound that activates δPKC will increase production of ROS. A compound that inhibits or antagonizes δPKC will decrease ROS generation. The δV1-1 inhibitor is merely exemplary of compounds that modulate ROS production.

The ability to modulate ROS production in cells is desirable since ROS have been implicated in the development of many disorders. ROS are involved in artherosclerotic lesions, in the evolution of various neurodegenerative diseases, and are also produced in association to epileptic episodes, in inflammation, in the mechanisms of action of various neurotoxicants, or as side effects of drugs. Thus, further studies were conducted to examine the role of δPKC in conditions involving ROS, such as arteriosclerosis.

Arteriosclerosis, or fibrotic hardening of the arteries, is a factor in the cardiovascular morbidity associated with several disease processes, including type 2 diabetes. Arterial stiffness, as measured by increased pulse-wave velocity, is an independent predictor of stroke, and is significantly associated with cardiovascular mortality independent of age. Arteriosclerosis is thought to be due to increased deposition of extracellular matrix proteins, as well as modification of matrix structure towards a less compliant phenotype. Calcification of vascular tissue may be due to an active morphogenetic process that also contributes to arteriosclerosis. Transforming Growth Factor-Beta-1, or TGF-Beta-1, is a cytokine known to induce fibrosis. TGF-beta signaling is transduced via intracellular Smad proteins. There may be some “cross-talk” between Smads and Core Binding Factor Alpha-1, or Cbfa-1, a transcription factor commonly associated with bone development, which itself can upregulate the expression of calcific proteins in the vasculature.

Hyperglycemia may be an inciting factor for diabetes-associated arteriosclerosis, however, the arteriosclerotic process may begin during the insulin-resistant, euglycemic state. Studies were done to examine the effect of the factors associated with insulin-resistance, with a focus on hyperinsulinemia, on the differential activation of PKC isoforms in vascular tissues. These studies also included an examination of whether changes in gene expression towards a pro-fibrotic phenotype were associated with activity of various PKC isoforms, and to what extent the effects seen could be via reactive oxygen species-dependent versus independent mechanisms. These studies will now be described.

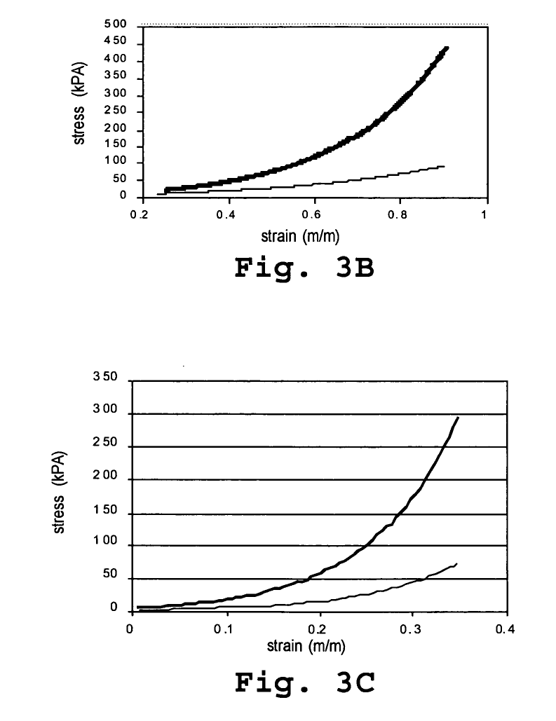

Zucker obese rats (fa/fa), which bear a mutation (fa) in the leptin receptor gene, provide a model of obesity associated with type 2 diabetes and typically exhibit pronounced hyperinsulinemia and hyperlipidemia at 6 weeks of age and became diabetic after 14 weeks of age. Tissue homogenates from the aortas of Zucker obese rats and of Zucker lean (Fa/fa) rats were prepared and analyzed for superoxide production using electron spin resonance with a tempomaine probe. As seen in FIG. 3A, the obese rats had a significantly higher production of ROS than their lean counterparts. Obese rats treated with insulin-sensitizing thiazolidinedione (TZD1) brought the ROS production down to control levels.

The isolated aortas were isolated from the animals and analyzed using a tissue mechanical tester, as described in Example 2, in both the circumferential direction and the longitudinal direction. FIGS. 3B-3C shows the stress-strain relationship in the circumferential direction and the longitudinal direction, respectively. The averages under the curve for each data set indicates increased developed strain in Zucker obese rats (upper curve) compared to Zucker lean rats (lower curve) in both the circumferential and longitudinal directions. Modeling this data with a soft tissue strain energy equation further demonstrated decreased anisotropy (i.e., varying tissue properties based on the direction of strain) in the Zucker obese rat aorta as a consequence of the greater increase in the longitudinal component than the circumferential component.

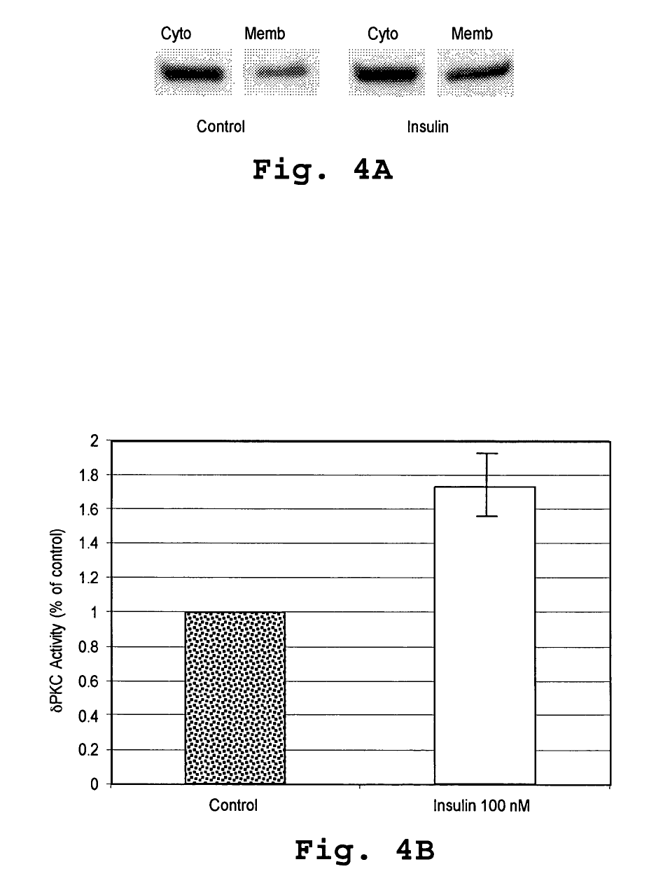

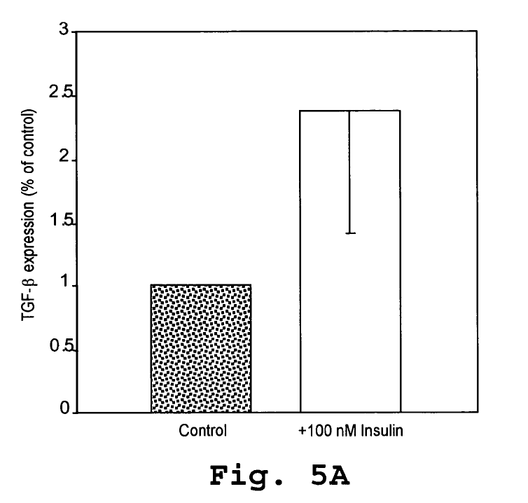

Example 3 describes a study conducted to examine to role of PKC isozymes, and in particular δPKC, in vascular fibrosis, which is accelerated during the insulin-resistant, pre-diabetic state in animals. In this study, rat vascular smooth muscle cells were incubated with bovine insulin. Translocation of PKC isozymes was assessed by Western blot analysis of the cystosolic and membrane (particulate) fractions of the cells. The subcellular localization of various PKC isozymes was assessed by immunofluorescence by probing the blot with isozyme specific antibodies, such as anti-δPKC, anti-αPKC, and anti-ε PKC, etc. The results for a blot probed with anti-δPKC antibodies are shown in FIGS. 4A-4B.

FIG. 4A shows the Western blot autoradiogram of the cytosol and membrane cell fractions of rat vascular smooth muscle cells incubated with bovine insulin and probed with anti-δPKC. Visual inspection of the figure and comparison to the control cells which were not incubated with insulin shows that δPKC is significantly stimulated by insulin. FIG. 4B is a bar graph showing the relative δPKC activity, based on the Western blot of FIG. 4A, of cells incubated with bovine insulin. This semi-quantitative representation of the blot shows that δPKC activity is stimulated by a factor of about 1.7 in the presence of insulin.

Accordingly, the methods described herein include a method for inhibiting δPKC activity (e.g., cellular translocation) to treat or to slow progression of arteriosclerosis in a person, particularly in a diabetic person.

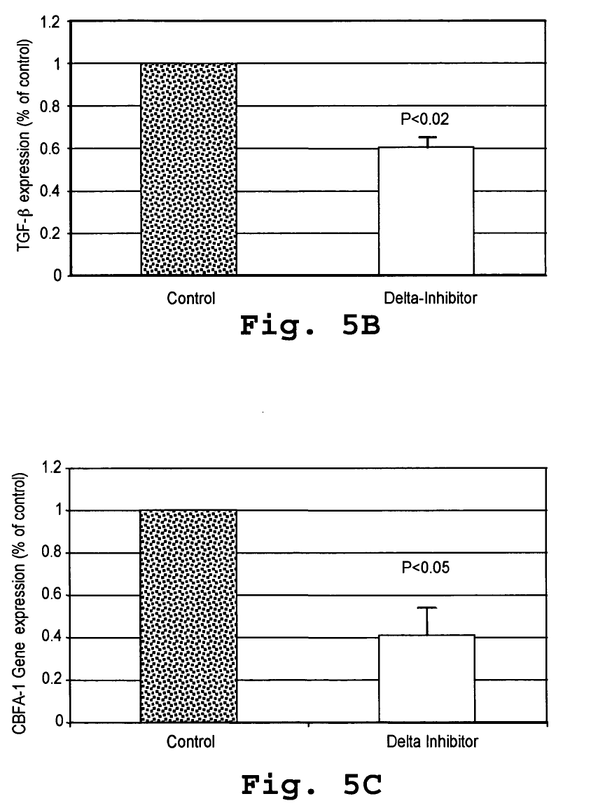

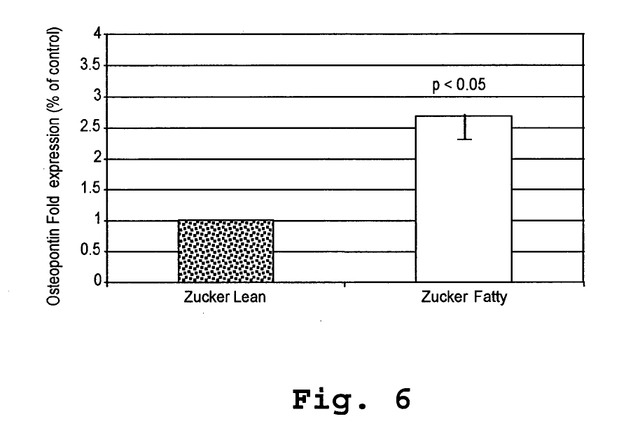

In another study, described in Example 4, the gene transcriptional effects of insulin and the selective inhibition of PKC isoforms was examined using real-time polymerase chain reaction (TaqMan® PCR). In vitro cells were incubated with insulin and in the presence or absence of a δPKC inhibitor, δV1-1 peptide. The expression of TGF-β and core binding factor α-1 (Cbf α-1) were monitored using TaqMan® real time PCR. The results are shown in FIGS. 5A-5C.

FIG. 5A is a bar graph showing the normalized TGF-β expression in vitro in rat vascular smooth muscle cells left untreated (control) and incubated with insulin. Insulin alone did not appear to stimulate the genes involved in vascular fibrosis, such as TGF-β1.

FIGS. 5B-5C show the effect of a δPKC inhibitor compound on expression of TGF-β1 and Cbfa-1. In FIG. 5B, the TGF-β1 expression is inhibited when cells are incubated with Tat-conjugated δV1-1peptide. Similarly, as seen in FIG. 5C, Cbfa-1 gene expression in vitro is inhibited with cells are incubated with Tat-conjugated δV1-1 peptide.

In summary, these studies show that inhibition of δPKC was associated with a significant decrease in the expression of TGF-β1 and Cbfa-1. δPKC also decreased the mRNA expression of PAI-1, extracellular matrix proteins such as fibronectin and collagen 4alpha3, and pro-calcific proteins such as osteopontin (data not shown). Expression of TGF-β1 is involved in sclerotic processes, including myocardial fibrosis, diabetic nephropathy, and scleroderma. Overexpression leads to excessive matrix deposition and fibrosis of certain organs. Accordingly, it will be appreciated that administering a compound that inhibits δPKC activity in a cell decreases expression of factors involved in, for example, arteriosclerosis, and other disorders associated with increased TGF-β1 and Cbfa-1 expression, such as inflammation.

In another study, described in Example 5, aortas were isolated from obese and lean Zucker rats. RNA isolated from the aortas were analyzed for expression of osteopontin. FIG. 6 is a bar graph showing the increase in osteopontin expression in vivo in Zucker obese rats relative to Zucker lean rats. The isolated aortas were also tested for elasticity of the vessel, and as FIG. 3B shows, aortas excised from older Zucker obese rats demonstrated increased wall strain compared to aortas of age-matched lean Zucker rats. The data demonstrating increased vascular stiffness in the insulin-resistant phenotype, ex-vivo, confirms that the fibrotic processes seen at the mRNA and protein level has pathophysiological consequences.

### III. TREATMENT METHODS

As described above, the peptides δV1-1, δV1-2, δV1-5, and ψδRACK act as translocation inhibitors or activators of δPKC. ψδRACK is an agonist, inducing translocation of δPKC to promote production of ROS. δV1-1, δV1-2, and δV1-5 are antagonists, inhibiting δPKC translocation and inhibiting production of ROS. Thus, methods of modulating the level of ROS in a cell or tissue by administering compounds that modulate appropriately the activity of δPCK are contemplated.

It will be appreciated that administration of a peptide having inhibitory or stimulatory activity on δPKC is merely exemplary of the general concept of administration of any compound having inhibitory or stimulatory activity on δPKC. Those of skill in the art will understand that compounds having such activity are known or can be identified, and can include organic compounds that act as peptidomimetics.

The method of the invention is intended for use in treating any condition associated with oxidative stress, e.g., any disorder characterized by or preceded by an increased level of ROS. The method also finds use in cancer therapy, where the level of ROS in tumor cells is increased by administration of a δPKC a compound that activates δPKC to increase superoxide and other ROS intracellularly. Conditions associated with increased levels of ROS, where a method of decreasing the level of ROS by administering a compound effective to inhibit δPKC activity, include, but are not limited to vascular disorders, such as vascular fibrosis, artherosclerotic lesions, etc., various neurodegenerative diseases, disorders characterized by inflammation, such as arthritis, inflammatory bowel disease, etc., kidney disease, cardiovascular disease, and general tissue damage.

In embodiments were a peptide is administered it will be appreciated that the peptides can be used in native form or modified by conjugation to a carrier, such as those described above. In one embodiment, the peptide is modified to include a terminal cysteine residue for attachment to a cell-permeable carrier peptide. It will also be appreciated that one or two amino acids from the sequences can be substituted or deleted while still retaining the desired peptide activity. Exemplary modifications for some of the peptides described above are given below.

For the ψδRACK peptide, identified as SEQ ID NO:5, potential modifications include the following changes shown in lower case: MkAAEDPM (SEQ ID NO:10), MRgAEDPM (SEQ ID NO:11), MRAgEDPM (SEQ ID NO:12), MRApEDPM (SEQ ID NO:13), MRAnEDPM (SEQ ID NO:14), MRAAdDPM (SEQ ID NO:15), MRAAEDPv (SEQ ID NO:16), MRAAEDPi (SEQ ID NO:17), MRAAEDPI (SEQ ID NO:18), and MRAAEDmp (SEQ ID NO:21), MeAAEDPM (SEQ ID NO:22), MdAAEDPM (SEQ ID NO:23), MRAAEePI (SEQ ID NO:24), MRAAEDPI (SEQ ID NO:25), MRAAEePi (SEQ ID NO:26), MRAAEePv (SEQ ID NO:27), MRAAEDPv (SEQ ID NO:28), and any combination of the above. The following modifications to ψδRACK are also contemplated and are expected to convert the peptide from agonist to an antagonist: MRAAnDPM (SEQ ID NO:29), and MRAAqDPM (SEQ ID NO:30), MRAAEqPM (SEQ ID NO:31), MRAAEnPM (SEQ ID NO:32). Suitable fragments of ψδRACK are also contemplated, and SEQ ID NOS: 19, 20 are exemplary.

Accordingly, the term “a δPKC agonist” as used herein intends a ψδRACK peptide, which refers to SEQ ID NO:5 and to peptides having a sequence homologous to SEQ ID NO:5 and to peptides identified herein, but not limited to, as SEQ ID NO: 10-18 and SEQ ID NO:20-28. The term a δPKC agonist further refers to fragments of these ψδRACK peptides, as exemplified by SEQ ID NOS:19-20.

For δV1-1, potential modifications include the following changes shown in lower case: tFNSYELGSL (SEQ ID NO:33), aFNSYELGSL (SEQ ID NO:34), SFNSYELGtL (SEQ ID NO:35), including any combination of these three substitutions, such as tFNSYELGtL (SEQ ID NO: 36). Other potential modifications include SyNSYELGSL (SEQ ID NO:37), SFNSfELGSL (SEQ ID NO:38), SNSYdLGSL (SEQ ID NO:39), SFNSYELpSL (SEQ ID NO:40). Other potential modifications include changes of one or two L to I or V, such as SFNSYEiGSv (SEQ ID NO:41), SFNSYEvGSi, (SEQ ID NO:42) SFNSYELGSv (SEQ ID NO:43), SFNSYELGSi (SEQ ID NO:44), SFNSYEiGSL (SEQ ID NO:45), SFNSYEvGSL (SEQ ID NO:46), aFNSYELGSL (SEQ ID NO:47), and any combination of the above-described modifications. Fragments and modification of fragments of δV1-1 are also contemplated, such as YELGSL (SEQ ID NO:48), YdLGSL (SEQ ID NO:49), fdLGSL (SEQ ID NO:50), YdiGSL (SEQ ID NO::51), YdvGSL (SEQ ID NO:52), YdLpSL (SEQ ID NO:53), YdLgIL (SEQ ID NO:54), YdLGSi (SEQ ID NO:55), YdLGSv (SEQ ID NO:56), LGSL (SEQ ID NO:57), iGSL (SEQ ID NO:58), vGSL (SEQ ID NO:59), LpSL (SEQ ID NO:60), LGIL (SEQ ID NO:61), LGSi (SEQ ID NO:62), LGSv (SEQ ID NO:63).

Accordingly, the term “a δV1-1 peptide” as used herein refers to an isolated peptide identified by SEQ ID NO:3 and to peptides having at least about 50% sequence identity, more preferably 70% or 75% sequence identity, still more preferably 80% sequence identity, even still more preferably 90% or 95% sequence identity to SEQ ID NO:3, including but not limited to the peptides set forth in SEQ ID NOS:33-47, as well as fragments of any of these peptides that retain activity, as exemplified by but not limited to SEQ ID NOS:48-63.

For δV1-2, potential modifications include the following changes shown in lower case: ALsTDRGKTLV (SEQ ID NO:64), ALTsDRGKTLV (SEQ ID NO:65), ALTTDRGKsLV (SEQ ID NO:66), and any combination of these three substitutions, ALTTDRpKTLV (SEQ ID NO:67), ALTTDRGrTLV (SEQ ID NO:68), ALTTDkGKTLV (SEQ ID NO:69), ALTTDkGkTLV (SEQ ID NO:70), changes of one or two L to I, or V and changes of V to I, or L and any combination of the above. In particular, L and V can be changed to V, L, I R and D, E can change to N or Q.

Accordingly, the term “a δV1-2 peptide” as used herein refers to an isolated peptide identified by SEQ ID NO:4 and to peptides having at least about 50% sequence identity, more preferably 70% or 75% sequence identity, still more preferably 80% or 85% sequence identity, even still more preferably 90% or 95% sequence identity to SEQ ID NO:4, including but not limited to the peptides set forth in SEQ ID NOS:64-70, as well as fragments of any of these peptides that retain activity.

For δV1-5 (SEQ ID NO: 6), potential modifications include those similar to the modifications described for δV1-2. The term “a δV1-5 peptide” as used herein refers to isolated peptides that retain the desired activity and have at least about 50% sequence identity, more preferably 70% or 75% sequence identity, still more preferably 80% or 85% sequence identity, even still more preferably 90% or 95% sequence identity to SEQ ID NO:6.

Accordingly, the term “a δPKC peptide antagonist” as used herein intends an isolated δPKC peptide, exemplified herein by a δV1-1 peptide, a δV1-2 peptide and a δV1-5 peptide.

In still other embodiments, the peptide can be part of a fusion protein or a transport protein conjugate. Typically, to form a fusion protein, the peptide is bound to another peptide by a bond other than a Cys-Cys bond. An amide bond from the C-terminal of one peptide to the N-terminal of the other is exemplary of a bond in a fusion protein. The second peptide to which the δPKC agonist/antagonist peptide is bound can be virtually any peptide selected for therapeutic purposes or for transport purposes. For example, it maybe desirable to link the δV1-1 peptide to a cytokine or other peptide that elicites a biological response.

Where the peptide is part of a conjugate, the peptide is typically conjugated to a carrier peptide, such as Tat-derived transport polypeptide (Vives et al. J. Biol. Chem., 272:16010-16017 (1997)), polyarginine (Mitchell et al., J. Peptide Res., 56:318-325 (2000); Rothbard et al., Nature Med., 6:1253-1257 (2000)) or Antennapedia peptide by a Cys-Cys bond. See U.S. Pat. No. 5,804,604. As noted above, the transport peptide can be attached to the C-terminus or the N-terminus of the peptide. In another general embodiment, the peptides can be introduced to a cell, tissue or whole organ using a carrier or encapsulant, such as a liposome in liposome-mediated delivery.

The peptide may be (i) chemically synthesized or (ii) recombinantly produced in a host cell using, e.g., an expression vector containing a polynucleotide fragment encoding said peptide, where the polynucleotide fragment is operably linked to a promoter capable of expressing mRNA from the fragment in the host cell.

It will be appreciated that the dose of peptide administered will vary depending on the condition of the subject, the timing of administration (that is, whether the peptide is administered to prevent an increase in ROS, to treat an existing increase in ROS). Those of skill in the art are able to determine appropriate dosages, using, for example, the dosages used in in vitro and animal studies.

The peptides can be administered to the cell, tissue or whole organ in vitro, in vivo, or ex vivo. All modes of administration are contemplated, including intraveneous, parenteral, subcutaneous, inhalation, intranasal, sublingual, mucosal, and transdermal. A preferred mode of administration is by infusion or reperfusion through arteries to a target site. It will also be appreciated that a suitable pharmaceutical preparation can be prepared based on the selected route of administration. For example, the peptide can be delivered in a saline-based formulation. Other pharmaceutically-acceptable carriers, vehicles, and excipients are well known in the art and literature sources describing preparations for in vivo administration are readily available.

It will also be appreciated that any of the embodiments described here, preferred to non-preferred, can be combined with any other embodiment, preferred to non-preferred.

### V. EXAMPLES

The following examples further illustrate the invention described herein and are in no way intended to limit the scope of the invention.

### Example 1

### Production of Reactive Oxygen Species In Vitro

A. Peptide Preparation

δV1-1 (SEQ ID NO:3) and ψδRACK (SEQ ID NO:5) were synthesized and purified (>95%) at the Stanford Protein and Nucleic Acid Facility. The peptides were modified with a carrier peptide by cross-linking via an N-terminal Cys-Cys bond to Tat (SEQ ID NO:8).

B. Peptide Delivery into Cells

Rat vascular smooth muscle cells were harvested from Zucker lean aortas by enzymatic digestion and cultured in Dulbecco's modified eagle's medium with 10% fetal bovine serum and penicillin/streptomycin under standard tissue culture conditions. The cells were incubated in the presence tempoamine ((2,2,6,6-tetramethyl-1-piperidi-nyloxy4-amine)) (100 μM) and in the presence of (1) the agonist Tat-conjugated peptide ψδRACK (10 μM); (2) phorbol 12-myristate 13-acetate (PMA, 500 ng/mL); or (3) the antagonist Tat-conjugated peptide δV1-1 (10 μM) plus PMA (500 ng/mL). A control cell sample was left untreated. The loss of tempoamine signal was measured by electron spin resonance to measure superoxide production. The results are shown in FIG. 2.

### Example 2

### Mechanical Testing of Tissue

Seven male Zucker lean and eight Zicker obese (fa/fa, Harlan) rats were maintained on a normal chow diet and housed in a room with a 12-hour light/12-hour dark cycle. Aortas were harvested and snap-frozen in liquid nitrogen for subsequent RNA isolation while a subset were fixed in a 10% formalin for histological evaluation.

Aortic tissue mechanics were analyzed using a custom-made tissue mechanical tester. Thoracic aorta segments were chosen that had minimal branching vessels, and any branding vessels were ligated prior to explant. Within 4 hours of explant, 15 mm-long cylindrical thoracic aorta specimens were cannulated to barbed-end fittings with suture. Under constant longitudinal strain, held at in vivo length, pressure was gradually increased from 0-330 mmHg. Then, strain, held at mean physiological pressure, length was extended from relaxed to 150% of in vivo length. Constant pressure was maintained by using a large reservoir held approximately 6.5 feet above the specimen as a pressure source, making any leakage out of the explanted vessel negligible. Intraluminal pressure and axial load were recorded through a data acquisition system and correlated to regional tissue strain via video images. From this information, stress-strain relationships were computed. The isotropy index was computed using Fung's soft tissue strain energy equation (Fung, Y. C. et al., Am. J. Physiol., 237(5):H620-H631 (1979)). Results are shown in FIGS. 3B-3C.

### Example 3

### Translocation of δPKC in Cells Incubated with Insulin

Rat vascular smooth muscle cells passages three through seven were prepared as described in Example 1 B. The cells were incubated with bovine insulin (100 nM) for time periods of up to 24 hours. Activation of various PKC isozymes was analyzed by harvesting the cells and separating the protein into cytosolic and membrane (particulate) fractions and examining both fractions by Western blot using anti-δPKC, anti-αPKC, and anti-ε PKC for the presence of different PKC isoforms. The activity of the isoform was assessed as amount of PKC in the membrane fraction divided by the total amount of protein in both fractions (cytosol and membrane), normalized to control. The results are shown in FIGS. 4A-4B.

### Example 4

### Gene Transcriptional Effects of Insulin in vitro

Rat vascular smooth muscle cells passages three through seven were prepared as describe in Example 1B. The cells were incubated with insulin (100 nm, SourceLife Technologies) or with the δPKC inhibitor peptide δV1-1, prepared as described in Example 1A. RNA isolation and purification were performed as described in Example 2 and TaqMan® real time PCR was used to analyze the relative quantity TGF-β1 and core binding factor α-1 (Cbf α-1). PCR primers and probes were obtained commercially from Applied Biosystems, Inc. and the PCR was performed on an Applied Biosystems ABI Prism 7700. The results are shown in FIGS. 5A-5C.

### Example 5

### Expression of Factors Associated with Vascular Fibrosis in Zucker Obese and Lean Rats

Aortas from Zucker obese rats and Zucker lean (Fa/fa) rats, 10-16 weeks of age, were isolated. RNA from the aortas was analyzed for expression of osteopontin according to the methods described in Examples 2 and 4 above. The aortas were also tested for elasticity via mechanical testing of stress-strain, as described in Example 2 above. The results are shown in FIG. 6 and FIG. 3B.

Although the invention has been described with respect to particular embodiments, it will be apparent to those skilled in the art that various changes and modifications can be made without departing from the invention.

1. A method of modulating production of reactive oxygen species in a cell or tissue, comprising
administering a compound effective to modulate the activity of δPKC.

2. The method according to claim 1, wherein said compound has activity to inhibit δPKC activity to reduce production of reactive oxygen species in said cell or tissue.

3. The method according to claim 1, wherein said compound has activity to activate δPKC activity to stimulate production of reactive oxygen species in said cell or tissue.

4. The method according to claim 2, wherein said compound is a peptide having at least 50% sequence identity to a peptide selected from the group consisting of δV1-1 (SEQ ID NO:3), δV1-2 (SEQ ID NO:4), and δV1-5 (SEQ ID NO:6).

5. The method according to claim 4, wherein said peptide is selected from the group consisting of SEQ ID NO:3 ( δV1-1) and the peptides identified as SEQ ID NOS:33-47.

6. The method according to claim 4, wherein said peptide is selected from the group consisting of δV1-2 (SEQ ID NO:4) and the peptides identified as SEQ ID NOS:64-70.

7. The method according to claim 3, wherein said compound is a peptide having at least 50% sequence identity to ψδRACK (SEQ ID NO:5).

8. The method according to claim 7, wherein said peptide is selected from the group consisting of ψδRACK (SEQ ID NO:5) and the peptides identified as SEQ ID NOS:10-18, 21-32.

9. The method according to claim 4 wherein the peptide is linked to a moiety effective to facilitate transport across a cell membrane.

10. The method according to claim 9, wherein said moiety is a Tat-derived peptide.

11. A method for treating or slowing the progression of arteriosclerosis, comprising
administering a compound effective to inhibit the activity of δPKC.

12. The method according to claim 11, wherein said compound is a peptide having at least 50% sequence identity to a peptide selected from the group consisting of δV1-1 (SEQ ID NO:3), δV1-2 (SEQ ID NO:4), and δV1-5 (SEQ ID NO:6).

13. The method according to claim 12, wherein said peptide is conjugated to Tat.

14. A method for treating a disorder preceded by an increased production of reactive oxygen species in a cell or tissue, comprising
administering a compound effective to inhibit the activity of δPKC.

15. The method according to claim 14, wherein said compound is a peptide having at least 50% sequence identity to a peptide selected from the group consisting of δV1-1 (SEQ ID NO:3), δV1-2 (SEQ ID NO:4), and δV1-5 (SEQ ID NO:6).

16. The method according to claim 12, wherein said peptide is conjugated to Tat.

17. The method according to claim 14, wherein said disorder is an inflammatory disorder.

18. The method according to claim 17, wherein said inflammatory disorder is associated with the vasculature.

19. The method according to claim 17, wherein said inflammatory disorder is arthritis.

20. A method for stimulating production of reactive oxygen species in a cell or tissue, comprising
administering a compound effective to agonize the activity of δPKC.

21. The method according to claim 20, wherein said compound is a peptide having at least 50% sequence identity to ψδRACK (SEQ ID NO:5).

22. The method according to claim 21, wherein said peptide is conjugated to Tat.

23. The method according to claim 20, wherein said cell is a tumor cell.


SEQUENCE LISTING (71 sequences)

SEQ ID NO: 1 - protein, 141 aa, Rattus norvegicus
MAPFLRISFNSYELGSLQAEDDASQPFCAVKMKEALTTDRGKTLVQKKPTMYPEWKSTFDAHIYEGRVIQIVLMRAAEDPMSEVTVGVSVLAERCKKNNGKAEFWLDLQPQAKVLMCVQYFLEDGDCKQSMRSEEEAMFPT

SEQ ID NO: 2 - protein, 124 aa, Mus musculus
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLE

SEQ ID NO: 3 - protein, 10 aa, Rattus norvegicus
SFNSYELGSL

SEQ ID NO: 4 - protein, 10 aa, Rattus norvegicus
ALTTDRGKLV

SEQ ID NO: 5 - protein, 8 aa, Rattus norvegicus
MRAAEDPM

SEQ ID NO: 6 - protein, 58 aa, Rattus norvegicus
PFRPKVKSPRDYSNFDQEFLNEKARLSYSDKNLIDSMDQSAFAGFSFVNPKFEHLLED

SEQ ID NO: 7 - protein, 17 aa, Artificial Sequence
  Drosophila antennapedia homeodomain-derived carrier peptide
CRQIKIWFQNRRMKWKK

SEQ ID NO: 8 - protein, 10 aa, Artificial Sequence
  Tat-derived carrier peptide
YGKKRRQRRR

SEQ ID NO: 9 - protein, 6 aa, Artificial Sequence
  beta-PKC-selective activator peptide
SVEIWD

SEQ ID NO: 10 - protein, 8 aa, Artificial Sequence
  modified pseudo-delta RACK peptide
MKAAEDPM

SEQ ID NO: 11 - protein, 8 aa, Artificial Sequence
  modified pseudo-delta RACK peptide
MRGAEDPM

SEQ ID NO: 12 - protein, 8 aa, Artificial Sequence
  modified pseudo-delta RACK peptide
MRAGEDPM

SEQ ID NO: 13 - protein, 8 aa, Artificial Sequence
  modified pseudo-delta RACK peptide
MRAPEDPM

SEQ ID NO: 14 - protein, 8 aa, Artificial Sequence
  modified pseudo-delta RACK peptide
MRANEDPM

SEQ ID NO: 15 - protein, 8 aa, Artificial Sequence
  modified pseudo-delta RACK peptide
MRAADDPM

SEQ ID NO: 16 - protein, 8 aa, Artificial Sequence
  modified pseudo-delta RACK peptide
MRAAEDPV

SEQ ID NO: 17 - protein, 8 aa, Artificial Sequence
  modified pseudo-delta RACK peptide
MRAAEDPI

SEQ ID NO: 18 - protein, 8 aa, Artificial Sequence
  modified pseudo-delta RACK peptide
MRAAEDPL

SEQ ID NO: 19 - protein, 4 aa, Rattus norvegicus
EDPM

SEQ ID NO: 20 - protein, 5 aa, Rattus norvegicus
AEDPM

SEQ ID NO: 21 - protein, 8 aa, Artificial Sequence
  modified pseudo-delta RACK peptide
MRAAEDMP

SEQ ID NO: 22 - protein, 8 aa, Artificial Sequence
  modified pseudo-delta RACK peptide
MEAAEDPM

SEQ ID NO: 23 - protein, 8 aa, Artificial Sequence
  modified pseudo-delta RACK peptide
MDAAEDPM

SEQ ID NO: 24 - protein, 8 aa, Artificial Sequence
  modified pseudo-delta RACK peptide
MRAAEEPL

SEQ ID NO: 25 - protein, 8 aa, Artificial Sequence
  modified pseudo-delta RACK peptide
MRAAEDPL

SEQ ID NO: 26 - protein, 8 aa, Artificial Sequence
  modified pseudo-delta RACK peptide
MRAAEEPI

SEQ ID NO: 27 - protein, 8 aa, Artificial Sequence
  modified pseudo-delta RACK peptide
MRAAEEPV

SEQ ID NO: 28 - protein, 8 aa, Artificial Sequence
  modified pseudo-delta RACK peptide
MRAAEDPV

SEQ ID NO: 29 - protein, 8 aa, Artificial Sequence
  modified pseudo-delta RACK peptide
MRAANDPM

SEQ ID NO: 30 - protein, 8 aa, Artificial Sequence
  modified pseudo-delta RACK peptide
MRAAQDPM

SEQ ID NO: 31 - protein, 8 aa, Artificial Sequence
  modified pseudo-delta RACK peptide
MRAAEQPM

SEQ ID NO: 32 - protein, 8 aa, Artificial Sequence
  modified pseudo-delta RACK peptide
MRAAENPM

SEQ ID NO: 33 - protein, 10 aa, Artificial Sequence
  modified delta V1-1 peptide
TFNSYELGSL

SEQ ID NO: 34 - protein, 10 aa, Artificial Sequence
  modified delta V1-1 peptide
AFNSYELGSL

SEQ ID NO: 35 - protein, 10 aa, Artificial Sequence
  modified delta V1-1 peptide
SFNSYELGTL

SEQ ID NO: 36 - protein, 10 aa, Artificial Sequence
  modified delta V1-1 peptide
TFNSYELGTL

SEQ ID NO: 37 - protein, 10 aa, Artificial Sequence
  modified delta V1-1 peptide
SYNSYELGSL

SEQ ID NO: 38 - protein, 10 aa, Artificial Sequence
  modified delta V1-1 peptide
SFNSFELGSL

SEQ ID NO: 39 - protein, 9 aa, Artificial Sequence
  modified delta V1-1 peptide
SNSYDLGSL

SEQ ID NO: 40 - protein, 10 aa, Artificial Sequence
  modified delta V1-1 peptide
SFNSYELPSL

SEQ ID NO: 41 - protein, 10 aa, Artificial Sequence
  modified delta V1-1 peptide
SFNSYEIGSV

SEQ ID NO: 42 - protein, 10 aa, Artificial Sequence
  modified delta V1-1 peptide
SFNSYEVGSI

SEQ ID NO: 43 - protein, 10 aa, Artificial Sequence
  modified delta V1-1 peptide
SFNSYELGSV

SEQ ID NO: 44 - protein, 10 aa, Artificial Sequence
  modified delta V1-1 peptide
SFNSYELGSI

SEQ ID NO: 45 - protein, 10 aa, Artificial Sequence
  modified delta V1-1 peptide
SFNSYEIGSL

SEQ ID NO: 46 - protein, 10 aa, Artificial Sequence
  modified delta V1-1 peptide
SFNSYEVGSL

SEQ ID NO: 47 - protein, 10 aa, Artificial Sequence
  modified delta V1-1 peptide
AFNSYELGSL

SEQ ID NO: 48 - protein, 6 aa, Rattus norvegicus
YELGSL

SEQ ID NO: 49 - protein, 6 aa, Artificial Sequence
  modified fragment of delta V1-1 peptide
YDLGSL

SEQ ID NO: 50 - protein, 6 aa, Artificial Sequence
  modified fragment of delta V1-1 peptide
FDLGSL

SEQ ID NO: 51 - protein, 6 aa, Artificial Sequence
  modified fragment of delta V1-1 peptide
YDIGSL

SEQ ID NO: 52 - protein, 6 aa, Artificial Sequence
  modified fragment of delta V1-1 peptide
YDVGSL

SEQ ID NO: 53 - protein, 6 aa, Artificial Sequence
  modified fragment of delta V1-1 peptide
YDLPSL

SEQ ID NO: 54 - protein, 6 aa, Artificial Sequence
  modified fragment of delta V1-1 peptide
YDLGLL

SEQ ID NO: 55 - protein, 6 aa, Artificial Sequence
  modified fragment of delta V1-1 peptide
YDLGSI

SEQ ID NO: 56 - protein, 6 aa, Artificial Sequence
  modified fragment of delta V1-1 peptide
YDLGSV

SEQ ID NO: 57 - protein, 4 aa, Rattus norvegicus
LGSL

SEQ ID NO: 58 - protein, 4 aa, Artificial Sequence
  modified fragment of delta V1-1 peptide
IGSL

SEQ ID NO: 59 - protein, 4 aa, Artificial Sequence
  modified fragment of delta V1-1 peptide
VGSL

SEQ ID NO: 60 - protein, 4 aa, Artificial Sequence
  modified fragment of delta V1-1 peptide
LPSL

SEQ ID NO: 61 - protein, 4 aa, Artificial Sequence
  modified fragment of delta V1-1 peptide
LGLL

SEQ ID NO: 62 - protein, 4 aa, Artificial Sequence
  modified fragment of delta V1-1 peptide
LGSI

SEQ ID NO: 63 - protein, 4 aa, Artificial Sequence
  modified fragment of delta V1-1 peptide
LGSV

SEQ ID NO: 64 - protein, 11 aa, Artificial Sequence
  modified delta V1-2 peptide
ALSTDRGKTLV

SEQ ID NO: 65 - protein, 11 aa, Artificial Sequence
  modified delta V1-2 peptide
ALTSDRGKTLV

SEQ ID NO: 66 - protein, 11 aa, Artificial Sequence
  modified delta V1-2 peptide
ALTTDRGKSLV

SEQ ID NO: 67 - protein, 11 aa, Artificial Sequence
  modified delta V1-2 peptide
ALTTDRPKTLV

SEQ ID NO: 68 - protein, 11 aa, Artificial Sequence
  modified delta V1-2 peptide
ALTTDRGRTLV

SEQ ID NO: 69 - protein, 11 aa, Artificial Sequence
  modified delta V1-2 peptide
ALTTDKGKTLV

SEQ ID NO: 70 - protein, 11 aa, Artificial Sequence
  modified delta V1-2 peptide
ALTTDKGKTLV

SEQ ID NO: 71 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD